(19) United States
(12) Patent Application Publication
(10) Pub. No.: US 2014/0081648 A1
(43) Pub. Date: Mar. 20, 2014

(54) Method for Managing Long-Term Care Facilities

(71) Applicants: John Mabry, Salt Lake City, UT (US); Michael Christopherson, Salt Lake City, UT (US); Tom Paget, Salt Lake City, UT (US); Melanie Matthews, Olympia, WA (US); Bart McFall, Salt Lake City, UT (US)

(72) Inventors: John Mabry, Salt Lake City, UT (US); Michael Christopherson, Salt Lake City, UT (US); Tom Paget, Salt Lake City, UT (US); Melanie Matthews, Olympia, WA (US); Bart McFall, Salt Lake City, UT (US)

(21) Appl. No.: 13/622,533

(22) Filed: Sep. 19, 2012

(51) Int. Cl. G06F 19/00; G06Q 50/22

(52) U.S. Cl. CPC G06F 19/327; G06Q 50/22
USPC 705/2

(57) ABSTRACT

A system and method for assisting in the management of a long-term care (LTC) facility is disclosed. The method includes the steps of receiving rules information about rules dealing with operating a LTC facility. The method also includes receiving resident data about the therapy and care needs of each resident of the LTC facility. Lastly, the method involves analyzing the resident data in light of the rules data and displaying on a user interface one or more notifications or alerts that identify one or more of the rules that may be triggered based on the resident data.

### BACKGROUND

1. Field of the Invention

This application relates generally to computer software. More specifically, this application relates to computer implemented methods and systems for managing and assisting in the management of long-term care facilities.

2. Background

Long-term care (LTC) facilities offer medical and non-medical services to people who cannot care for themselves for long periods. The aged, disabled, and children may be residents of such facilities where they receive room and board, physical therapy, speech therapy, occupational therapy, recreation, and social services. Some residents may also receive help with activities of daily living, such as assistance with eating, standing, dressing, and other basic functions. Nursing homes (or skilled nursing unit) and assisted living facilities are common examples of LTC facilities.

In the United States, LTC facilities receiving funding from Medicare or Medicaid are subject to numerous federal and state regulations as well as rules from the Center for Medicare and Medicaid Services (CMS). For example, federal regulations require a minimum data set (MDS) assessment for assessing all residents. MDS assessment is used to measure each resident's functional capabilities and needs. MDS results are used to identify the Resource Utilization Group (RUGs) of the resident, which is a metric used in determining the amount of Medicare reimbursements for care. To ensure compliance with these regulations, audits are performed by government surveyors.

Administrators of LTC facilities are faced with the daily management of resident's needs, compliance with federal and state regulations and CMS rules, and the management of facility staff and therapy staff. To coordinate the various operations and personnel of the LTC facility, the administrator may hold frequent staff meetings, even daily meetings, to evaluate the progress of the residents, the work of the staff, and to ensure regulatory compliance. Moreover, the administrator must ensure that care and therapy activities provided by the facility qualify for reimbursement to ensure that the facility is adequately funded. Recent health care changes have increased the number of regulations and made reimbursement qualifications more onerous. These regulation changes have increased the pressure on administrators to efficiently manage the staff in order to provide the care needed and to ensure the financial stability of their LTC facility.

### SUMMARY

The present invention has been developed in response to problems and needs in the field of LTC facility management. Thus, systems and methods are provided that can compile information about residents, staff, and regulations and provide this information to the administrator. Moreover, the systems and methods can analyze the compiled information to ensure that residents are receiving proper care, staff is optimally utilized, regulations and rules are satisfied, and/or the facility is operating within its available budget from reimbursements. The computer implemented systems and methods may also provide or suggest alerts, tips, tools, and training to the administrator and/or facility staff.

Accordingly, in some aspects of the invention, a computer implemented method for aiding in the management of a LTC facility includes the steps of receiving rules information (or data) about rules dealing with operating a LTC facility. The method also includes receiving resident data about the therapy and care needs of each resident of the LTC facility. Lastly, the method involves analyzing the resident data in light of the rules data and displaying on a user interface one or more notifications or alerts that identify one or more of the rules triggered based on the resident data. In some implementations, the rules data includes Medicare regulations, Medicaid regulations, and/or CMS rules.

In another aspect, a system for aiding in the management of a LTC facility includes a rules module, a resident care module, and an analysis module. The rules module is configured to receive and store rules data relating to rules for operating a LTC facility. The resident scheduling module is configured to receive, from a LTC computer system, stored resident data relating to the therapy and care needs of residents of the LTC facility. In addition, the analysis module is configured to analyze the rules data and the resident data to identify if one or more of the rules are being triggered, and suggests actions to remedy the situation.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 illustrates a representative system for implementing embodiments of the invention.

FIG. 2 illustrates a representative networked system configuration that may be used in association with embodiments of the present invention.

FIG. 3 illustrates a flowchart of a representative method for assisting in the management of a LTC facility.

FIG. 4 illustrates a representative user interface for a system of assisting in the management of a LTC facility.

FIG. 5 illustrates a representative resident list user interface for a system of assisting in the management of a LTC facility.

FIG. 6 illustrates a representative resident data user interface for a system of assisting in the management of a LTC facility.

FIG. 7 illustrates a representative resident data and data entry user interface for a system of assisting in the management of a LTC facility.

FIG. 8 illustrates a flowchart of a representative method for determining and suggesting a resident's planning RUGs level.

FIG. 9 illustrates a representative resident data entry user interface for calculating a planning RUGs level.

FIG. 10 illustrates a representative planning RUGs suggestion user interface for a system of assisting in the management of a LTC facility.

FIG. 11 illustrates a representative resident calendar user interface for a system of assisting in the management of a LTC facility.

FIG. 12 illustrates a flowchart of a representative method for analyzing the staffing level of a LTC facility.

FIG. 13 illustrates a flowchart of a representative method for simulating the operation of a LTC facility.

FIG. 14 illustrates a flowchart of a representative method for analyzing financial aspects of a LTC facility.

FIG. 15 illustrates a flowchart of a representative method preparing a pre-meeting agenda and post-meeting report.

### DETAILED DESCRIPTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention may be grouped into subheadings. The utilization of the subheadings is for convenience of the reader only and is not to be construed as limiting in any sense.

Various operations may be described as multiple discrete operations in turn, in a manner that may be helpful in understanding embodiments of the present invention; however, the order of description should not be construed to imply that these operations are order dependent.

The description may use the phrases “in an embodiment,” “in some embodiments,” “in some implementations,” or “in some instances,” which may each refer to one or more of the same or different embodiments. Furthermore, the terms “comprising,” “including,” “having,” and the like, as used with respect to embodiments of the present invention, are synonymous with the definition afforded the term “comprising.”

This application relates generally to computer software. More specifically, this application relates to methods for assisting in the management of LTC facilities. The present systems and methods can be used by LTC facility administrators to compile information about residents, staff, and regulations and provide this information to the administrator. Moreover, the systems and methods can analyze the compiled information to ensure that residents are receiving proper care, staff is optimally utilized, regulation and rules are satisfied, and/or that the facility is operating within its available budget from reimbursements. The computer implemented systems and methods may also provide or suggest alerts, tips, tools, and training to the administrator and/or facility staff.

### Representative Operating Environment

Some embodiments of the present systems and methods related to a website or user interface of, for example, a mobile application that accesses a website or Internet services. As used herein the term website refers to a secure user-accessible network site that implements the basic World Wide Web standards for the coding and transmission of hypertext documents. These standards currently include HTML (the hypertext markup language) and HTTP (the hypertext transfer protocol). Note that the term “site” is not intended to imply a single geographic location as a website or other network site can, for example, include multiple geographically distributed computer systems that are appropriately linked together.

As will be appreciated by one skilled in the art, the present invention may be embodied as a system, method, computer program product or any combination thereof. Accordingly, the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present invention may take the form of a computer program product embodied in any tangible medium of expression having computer usable program code embodied in the medium.

The invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Embodiments of the present invention embrace one or more computer-readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer-readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system. While embodiments of the invention embrace the use of all types of computer-readable media, certain embodiments as recited in the claims may be limited to the use of tangible, non-transitory computer-readable media, and the phrases “tangible computer-readable medium” and “non-transitory computer-readable medium” (or plural variations) used herein are intended to exclude transitory propagating signals per se.

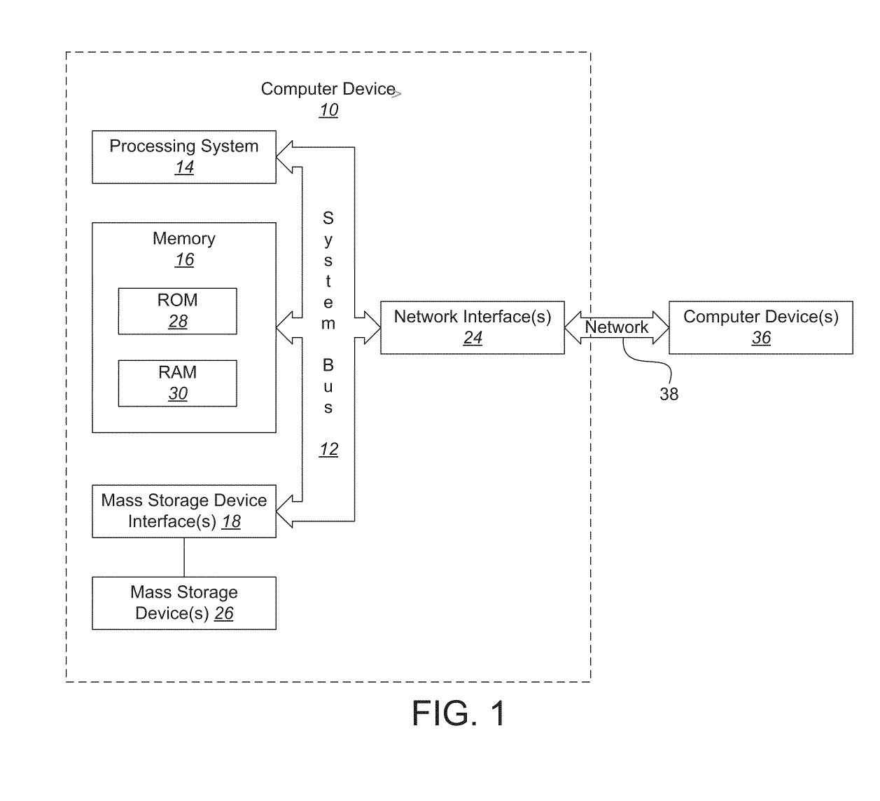

With reference to FIG. 1, a representative system for implementing embodiments of the invention includes computer device 10, such as one or more server system, a tablet computer or other mobile computer device, a general purpose computer, embedded systems with general purpose processing units, other stand alone electronic devices, and other such electronic environments. Computer device 10 may include a system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces, output interfaces, and/or network interfaces 24.

Processing system 14 includes one or more processors, such as a central processor and optionally one or more other processors designed to perform a particular function or task. It is typically processing system 14 that executes the instructions provided on computer-readable media, such as on memory 16, a magnetic hard disk, a removable magnetic disk, a magnetic cassette, an optical disk, or from a communication connection, which may also be viewed as a computer-readable medium.

Memory 16 includes one or more computer-readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include hard disk drives, magnetic disk drives, tape drives and optical disk drives. A mass storage device 26 may read from and/or write to a magnetic hard disk, a removable magnetic disk, a magnetic cassette, an optical disk, or another computer-readable medium. Mass storage devices 26 and their corresponding computer-readable media provide nonvolatile storage of data and/or executable instructions that may include one or more program modules such as an operating system, one or more application programs, other program modules, or program data. Such executable instructions are examples of program code means for implementing steps for methods disclosed herein.

One or more network interfaces 24 enable computer device 10 to exchange information with one or more other local or remote computer devices, illustrated as computer devices 36 (e.g., a mobile computing device such as a tablet computer or smart phone), via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, where functions or tasks are performed by a plurality of networked computer devices.

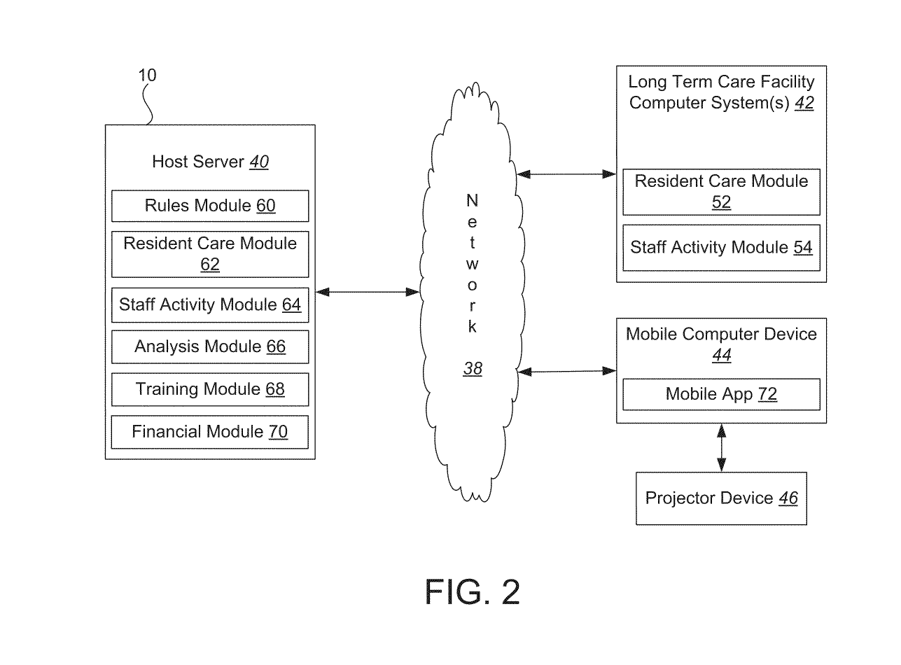

While those skilled in the art will appreciate that embodiments of the present invention may be practiced in a variety of different environments with many types of system configurations, FIG. 2 provides a representative networked system configuration that may be used in association with embodiments of the present invention. The representative system of FIG. 2 includes a computer device 10, illustrated as a host server 40, which is electronically coupled to one or more computer systems 42 of a LTC facility and a mobile computer device 44. While FIG. 2 illustrates an embodiment that includes a single host server 40, this post may include one or more servers working in combination. Other embodiments of the present invention include local, networked, or peer-to-peer environments where one or more computer devices may be connected to one or more local or remote peripheral devices. Moreover, embodiments in accordance with the present invention also embrace a wide area networked environments, such as the Internet.

Similarly, embodiments of the invention embrace cloud-based architectures where one or more computer functions are performed by remote computer systems and devices at the request of a local computer device. Cloud computing provides computation, software, data access and storage services that do not require end-user knowledge of the physical location and configuration of the system that delivers the services. Cloud computing encompasses any subscription-based or pay-per-use service and typically involves provisioning of dynamically scalable and often virtualized resources, including software as a service (SaaS) to other networked computer devices having a limited set of hardware and/or software resources. Cloud computing providers deliver applications via the internet, which can be accessed from a web browser, while the business software and data are stored on servers at a remote location. Moreover, the host server 40 can provide software and data support and services to mobile applications (“apps”) 72 of one or more mobile computer devices, or simply mobile devices, 44.

The mobile computer device (“mobile device”) 44 can be a two-way communication device having data communication capabilities. In addition, the device optionally has the capability to communicate with other computer systems via the Internet or another network 38. Note that the mobile device may comprise any suitable wired or wireless device such as multimedia player, mobile communication device, cellular phone, smartphone, PDA, PNA, Bluetooth device, tablet computing device such as the iPad, laptop, etc. The mobile device 44 may include a processing system (similar to processing system 14 of FIG. 1) and have operating system functions, which enable the execution of software applications on the mobile device 44. A predetermined set of applications that control basic device operations, such as data and voice communications, may be installed during manufacture. Additional applications, apps, may be downloaded from the Internet or another network source and installed in memory for execution on the processor. In some configurations, the mobile device 44 may be configured to communicate with a projector device 46 for displaying images, video, and other information.

Referring still to FIG. 2, the host server 40 can be in electronic communication with one or more computer systems 42 of a LTC facility to communicate data therebetween. LTC facilities offer medical and non-medical services to people who cannot care for themselves for long periods. Nursing homes (or skilled nursing unit) and assisted living facilities are common examples of LTC facilities. As will be understood, a LTC facility can include one or more computer systems 42 that can include resident care modules 52, and/or staff activity modules 54. These modules may each be contained on a single computer system or may be dispersed over one or more computer systems in various configurations. These modules will now be described.

In some embodiments, a computer system 42 of a LTC facility can include a resident care module 52. In some embodiments, this module can include one or more software programs (e.g., AHT software, HealthMEDX Software, etc.) that include records about individual residents. In the remainder of this specification, such software programs will be generally referred to as AHT. However, any similar software program for maintaining records about individual residents could also be used. As mentioned, LTC facilities offer medical and non-medical services to people who cannot care for themselves for long periods, such as the aged, disabled, and children. The LTC facility can provide the residents room and board, physical therapy (PT), speech therapy (ST), occupational therapy (OT), recreation, and social services. Residents may also receive help with activities of daily living, such as assistance with eating, standing, and dressing. Accordingly, resident information, or resident data, can include medical records, health records, therapy records, care needs, dietary restrictions, personal interests, hobbies, reasons for admittance, therapy programs, therapy schedules, medication schedules, care schedules, meal schedules, recreation schedules, records of care and treatment, Activity of Daily Living (ADL) scores, other records, and other such information. Specifically, resident data can identify the various therapy and care needs of each resident as well as provide a schedule for the delivery of the needed therapy and care. These software programs may include pre-existing software programs from a variety of vendors or software modules provided by the administrator of the host server 40 for transmitting information to the host server. Data about the residents, or resident data, can be input into, updated, stored, synchronized, and/or accessed via the resident care module 52.

Additionally, the LTC facility can include one or more computer systems 42 that include one or more staff activity modules 54. As will be understood, LTC facilities may employ a variety of full-time, part-time, or contract-based staff (herein simply “staff”) to meet the needs of the residents. Staff can include facility staff and therapy staff, such as director(s) of nursing, licensed practical nurses (LPNs), licensed vocational nurses (LVNs), physical therapists, occupational therapists, speech therapists, social service providers, recreational therapists/staff, case managers, nutrition and dietary specialists, outsourced therapist/staff, and staff assigned to provide help with activities of daily living. The one or more staff activity modules 54 can include one or more software programs or other modules configured to receive, update, and store the schedules, reports, and other activities of the staff. Some embodiments of the staff activity module 54 include staff note keeping or reporting software, timekeeping software (e.g., Kronos Timekeeper software), scheduling software, and/or other suitable staff management software. Information about staff activities, including care and therapy activities can be referred to as staff data.

Referring still to FIG. 2, in some embodiments, at least some of the resident data and staff data can be transmitted to the host server 40 via a network 38, such as the Internet. This information can be manually or automatically uploaded or synchronized. The host server 40 can utilize this data to assist in the management of the LTC facility. Similarly, the host server 40 can be configured to manually or automatically upload or synchronize resident data, staff data, and rules data to the LTC facility computer system(s) 42, as described below. Particular aspects of the host server 40 are described in detail in the section provided below.

### Representative Systems and Methods

The present systems and methods are utilized to assist in managing or in managing a LTC facility. This assistance can include compiling information about residents, staff, and regulations and providing this information to the administrator. Moreover, the systems and methods can analyze the compiled information to ensure that residents are receiving proper care, staff is optimally utilized, regulation and rules are satisfied, and/or the facility is operating within its available budget from reimbursements. The systems and methods may also provide or suggest alerts, tips, tools, and training to the administrator and/or facility staff.

Accordingly, referring again to FIG. 2, the host server 40 system (or simply system) can include multiple modules that are used to compile information about the management of the LTC facility that can be provided to, displayed on, and manipulated by the mobile device 44. As shown, in some embodiments, the host server 40 includes a general rules module 60, a resident care module 62, the staff activity module 64, an analysis module 66, a training module 68, and/or a financial module 70. Moreover, in some embodiments the rule module 60 can receive additional rules data from a server administrator or other third party. This rules data can include general rules relating to the operation of LTC facilities. For example, general rules data can include Medicare regulations, Medicaid regulations, and CMS rules. This information can be continually updated as these regulations and rules are amended. Moreover, the general rules can include information about insurance regulations, reimbursements, and policies. Furthermore, the general rules can include other government regulations or rules that are related to the regulation of businesses in general or specifically to LTC facilities. Because these rules can be complex, the present systems and methods can automatically apply these rules and regulations to the circumstances of a particular LTC facility to assist facility administrators in complying with these rules, as described with reference to the analysis module 66.

As further shown, the host server 40 can include a resident care module 62. The resident care module 62 can be configured to receive resident data from the resident care module 52. Resident data can include any information about a resident such as daily progress notes created by a nurse or other staff member for each resident. Resident data can include any information about a resident such as daily progress notes created by a nurse or other staff member for each resident. In some embodiments, if the resident data is modified by the mobile device 44, these changes can be updated on the resident care module 62. This ability can provide real-time, live resident information to users of the mobile device 44 and to the host server 40. The staff activity module 64 of the host server 40 can be configured to receive staff data from the staff activity module 54 of the computer systems 42 of the LTC facility.

Referring still to FIG. 2, the host server 40 can also include an analysis module 66. The analysis module 66 can be configured to analyze all or combinations of the rules data, the resident data, and/or the staff data to identify any of the rules that are triggered based on the resident data and/or the staff data. Various embodiments and examples of such analysis are described below. Generally, the analysis module 66 can identify instances where a resident's therapy or care activities fail to comply with one or more local or general rules. As noted above, these rules can include Medicare, Medicaid, CMS, insurance, and other applicable rules. Further still, the analysis module 66 can be configured to provide tips for optimally utilizing facility staff by increasing or decreasing staff size, using outsourced staff, and recognizing staff inefficiencies.

In some configurations, the host server 40 includes a training module 68. The training module 68 can coordinate with the analysis module 66 to provide training to the administrator or staff of a LTC facility in areas in which one or more rules may be triggered. Additionally or alternatively, the training module 68 can enable an administrator to simulate various management or operation scenarios relating to the management and operation of the LTC facility.

In some configurations, the host server 40 can also include a financial module 70. Financial module 70 can analyze the financial operations of LTC facility, including reimbursements, other income, and expenditures. This analysis can provide forecasts projections along with tips and notices to the administrator in order to assist him in operating the LTC facility.

FIG. 2 also illustrates a mobile device 44 that can incorporate a mobile application 72 that can be used to access, modify, and synchronize data with a system for assisting in the management of a long-term facility, such as the host server 40. In some instances, the mobile device 44 is part of this system. The mobile application 72 can be web-based, in which data presented by the mobile device 44 is primarily stored on the host server 40. In accordance with some embodiments, the mobile device 44 is adapted to implement the system as hardware, software or as a combination of hardware and software. In one embodiment, implemented as a software task, the program code operative to implement the present system is executed as one or more tasks running on one or more processors and stored in memory on the mobile device 44.

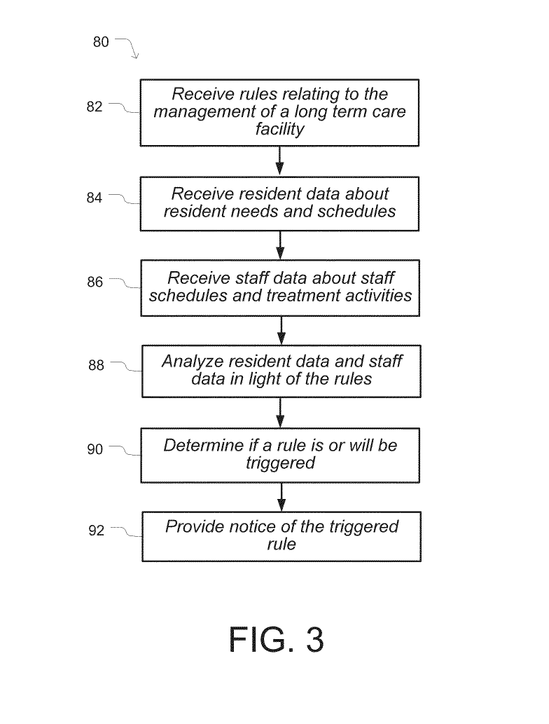

Reference will now be made to FIG. 3, which illustrates a representative method 80 for assisting in the management of a LTC facility. As indicated above, the method 80 can be implemented using a system, such as the host server (40 in FIG. 2) acting as a web server, and accessed using a computer device, such as the mobile device (44 in FIG. 2). In step 82, the method 80 receives rules relating to the management of a LTC facility. The rules can include the rules data previously mentioned, including local rules and general rules. In step 84, the method 80 can receive resident data about resident's needs and schedules. These needs can include therapy and care needs. In some embodiments, the method 80 can present and display resident data via a website or other user interface, as previously described. In step 86, the method 80 can optionally receive staff data about staff schedules and treatment activities. After receiving the resident data, and optionally the staff data, in step 88, the method 80 can analyze this data in light of the received rules. In step 90, the method 80 can determine if a rule is or will be triggered based on the resident data relating to the therapy and care of the resident and potentially based on the staff data. If it is determined that a rule is or will be triggered, in step 92 a notice is provided of the triggered rule. In some situations, such methods can assist an administrator to ensure compliance with the wide and complex insurance, Medicare, Medicaid, and other government rules and regulations.

In one or more embodiments of the invention, the determination of whether a rule is triggered or is likely to be triggered is made using a set of logic rules. The logic rules compare resident data (e.g. which treatments have been reported as having been provided) with the governing rules and regulations (e.g. Medicare/Medicaid rules, local rules, etc.) and generate notifications of triggered rules, potential triggered rules, or other type of warning or reminder regarding the input of resident data.

In this specification, triggered rule is used to refer to any type of non-compliance with any type of governing rule or recommendation. Accordingly, a triggered rule can refer to non-compliance with a requirement to receive reimbursement as well as to non-compliance with a standard of care that is required to be provided to a resident. In a particular example, the present invention identifies timing rules and provides notifications of requirements that must be met by a specified time. For example, the invention can provide notifications of approaching deadlines or past deadlines for filing certain required forms for receiving reimbursement for care provided to a patient.

For example, the logic rules can identify that some data required to receive reimbursement for a particular treatment has not been entered, and can display a warning or reminder that such data is required by a certain date in order to receive reimbursement for the treatment (i.e. certain electronic records must be completed and filed by a certain date in order to receive reimbursement for the care rendered). Similarly, the logic rules can identify that a resident should receive more care based on the patient's existing clinical care plan and can display a suggestion that such additional treatments should be considered.

In some embodiments, the logic rules are user configurable. For example, a system administrator can customize the logic rules to specify certain conditions for which notifications (e.g. of potential triggered rules, reminders, warnings, suggestions, etc.) will be generated, to specify custom notifications, or to create new logic rules to identify one or more other custom scenarios that a user may desire to monitor. In short, the logic rules allow the user to be continually informed of patient treatment or care plans that may fail to meet any number of governing rules or regulations, that may not comply with the LTC facility's policies or goals, that may be improved or enhanced, or that may be modified in any way.

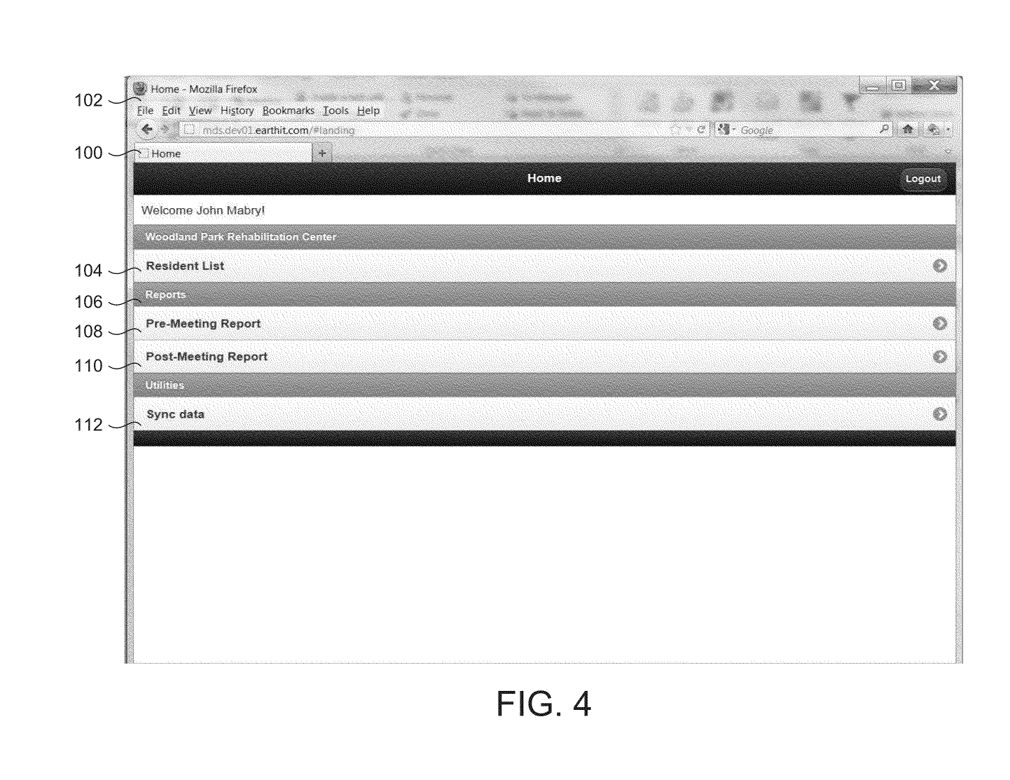

Reference will now be made to FIGS. 4 to 7, which illustrate various user interfaces that can implement one or more of the steps of method 80 of FIG. 3. Additionally, as will be seen, these user interfaces can provide various additional tools, features, and other resources for LTC facility administrators. Reference will first be made to FIG. 4, which illustrates a user interface 100, which is illustrated as a website, but which is not limited to a website accessed by a web browser 102. The user interface 100 provides resources for a LTC facility management system. It will be understood, that the user may log into the user interface 100 to search, view, upload, change, or delete data relating to the LTC facility that is stored on host system 40. It is noted that user interface 100 does not enable the user to modify data stored on LTC facility computer system 42 (e.g. in the AHT system). Such data in the AHT system can only be modified via the MDS process. In this process, the user interface 100 can provide information, tools, tips, alerts, and other material to the user.

The user interface 100 can include multiple sections, such as a resident's list section 104 where a user may see and modify system contents relating to facility residents, in accordance with their configured roles or permissions. In another section, a reports section 106, the reports are displayed, prepared, and submitted. These reports can include pre-meeting reports 108 and post meeting reports 110. Note that the system may be operative to provide an interactive environment on mobile (portable) devices for viewing information about each resident in a LTC facility.

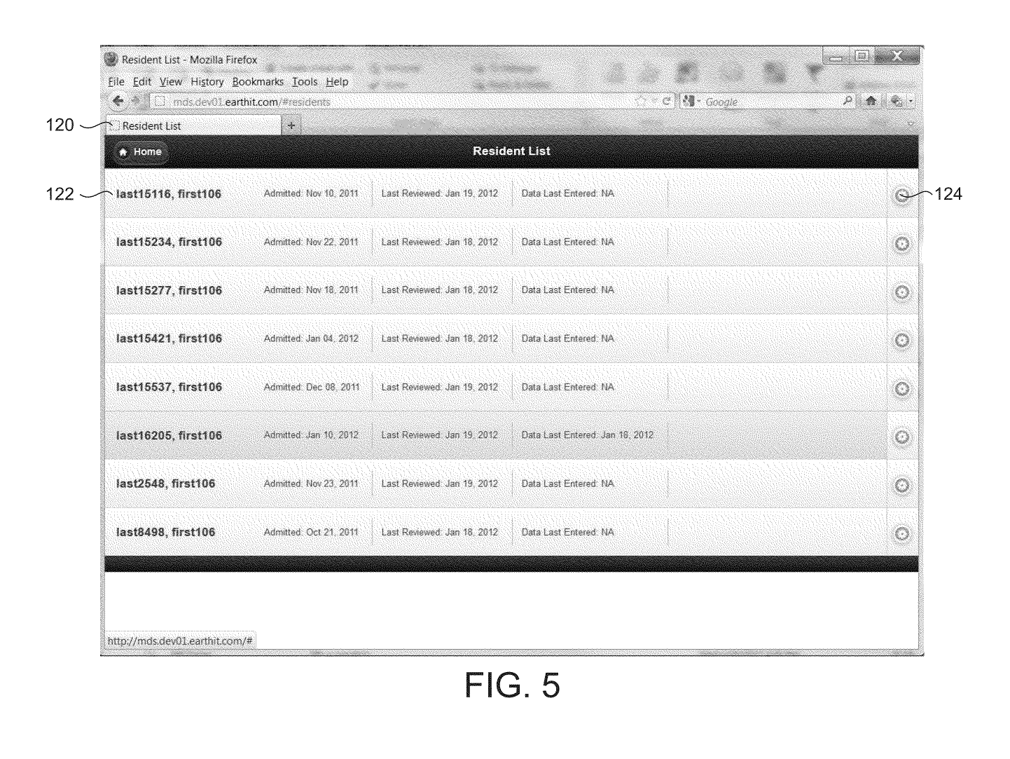

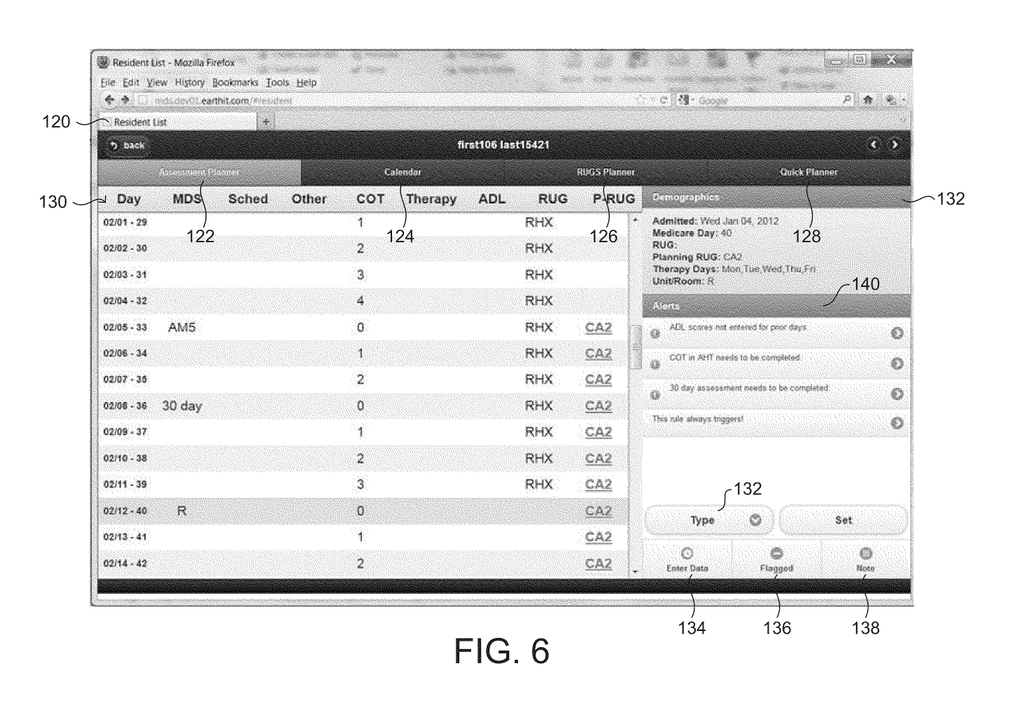

As shown in FIG. 5, by selecting the resident list section 104, in FIG. 4, a residents list 120 is presented to the user. This list can include the names 122 of each resident. When the name or another icon 124 is selected, resident data for that resident can be presented on the separate user interface, as shown in FIG. 6.

A resident data site, such as that shown in FIG. 6, can allow the user to access an assessment planner 122, a calendar 124 of the resident's activities, and/or a care coordination planner 128. The illustrated assessment plan 122 provides an interface 130 that has tools for planning the assessment of the particular resident. For example, as shown, for each day the following information can be provided, an assessment from AHT (i.e. from LTC Facility Computer System 42) or from the resident needs module (52 or 62 of FIG. 2) (MDS), a schedule (Sched), therapy observation periods (COT), minutes in therapy (Therapy), a daily “Activity of Daily Living” or ADL score (ADL), a Resource Utilization Group (RUGs) level from AHT or from the resident needs module (RUGs), and a care planning RUGs level (P-RUGs). In other embodiments, the assessment planner 122 can include other categories. Additionally, the assessment planner 122 can display the demographics 132 of the resident. Moreover, one or more features can be provided for entering data 132, for flagging the assessment 136, for adding notes 138, or for selecting the assessment type 132.

As previously mentioned with reference to FIG. 3, in some embodiments, the system can analyze the resident data in light of general and/or local rules relating to the operation of the LTC facility. When it is determined via the logic rules that a rule is or will be triggered or that a notification, warning, or suggestion should be provided, the system can notify the user or administrator accordingly. For example, in some embodiments shown in FIG. 6, the system displays a notice to the user of such triggered rules in the form of one or more alerts 140. Some alerts 140 can indicate to the user instances in which the planned assessment or prior assessments of the resident have failed to comply with a rule. For example, as shown, one alert indicates that the ADL score was not entered for prior days. In another example an alert indicates that the COT in AHT needs to be completed. Another alert indicates that a 30-day assessment needs to be completed. These alerts can identify breaches in the rules required for reimbursement for the assessed activities and therapies. In some instances, failing to attend to these alerts can lead to a failure of reimbursement for therapies provided to the resident. Because there are so many types of rules governing LTC facilities, it will be understood that the system can identify a variety of discrepancies in the nature of the recording of therapies, the type of therapy provided to a resident, the frequency of the therapy provided to the resident, care provided to the resident, or various other discrepancies that are out of compliance with any rule or company policy. In some embodiments, the alert or notice provides a link that brings up a user interface where the user can take appropriate action to correct the failure that resulted in the triggered rule.

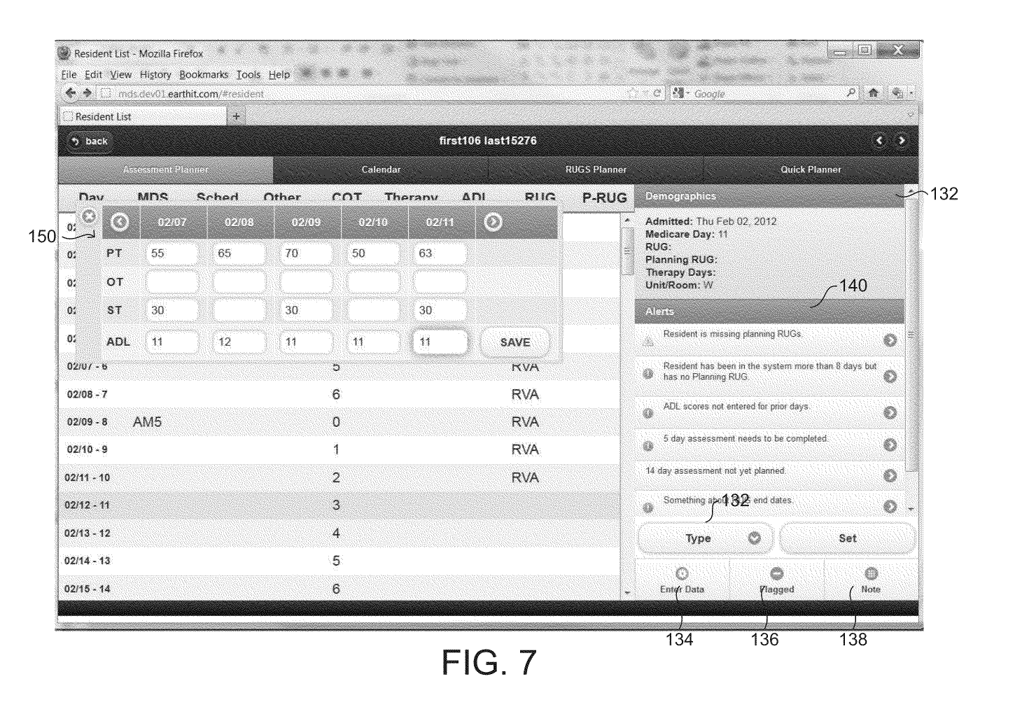

In some instances, to correct triggered rules, additional recordkeeping or information needs to be submitted to the system. Accordingly as shown in FIG. 7, data can be entered relating to the therapy of the resident via an interface 150. For example, such interfaces can record the minutes in which the resident engaged in his physical therapy (PT), occupational therapy (OT), speech therapy (ST), along with the ADL of the resident for a given day. It is again noted that this input is recorded in host server 40, but is not propagated to LTC Facility Computer System 42 (e.g. to modify the AHT system) because the data in LTC Facility Computer System 42 is set and modified via the MDS process.

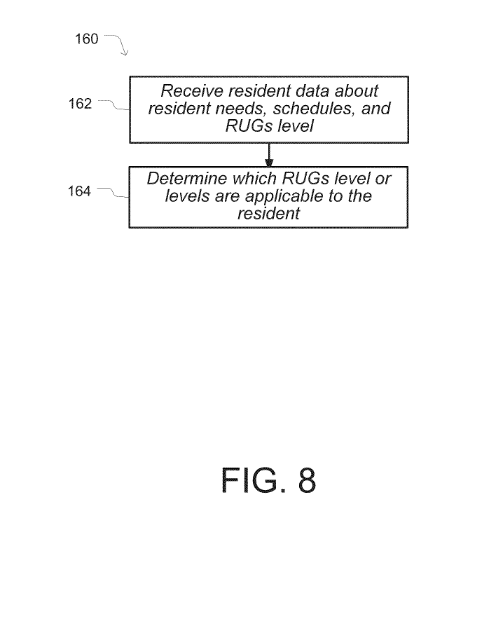

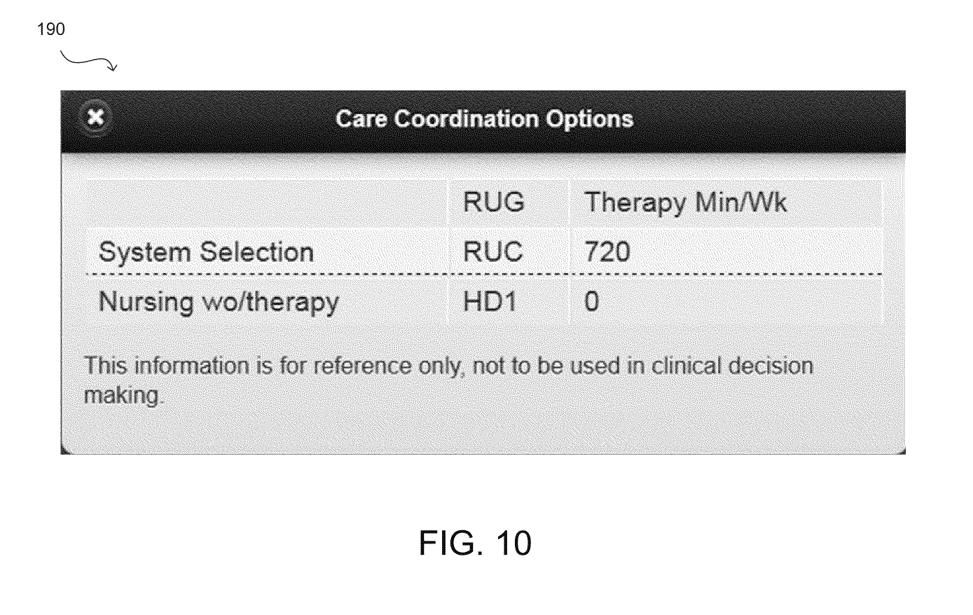

Reference will now be made to FIG. 8. As previously mentioned, rules data may include Medicare regulations, Medicaid regulations, and CMS rules. Specifically, the rules data may include RUGs level standards and the resident data includes a current RUGs level based on the MDS assessment. In order to deliver the level of care agreed upon by the Interdisciplinary clinical team in the clinical care plan, it is important to maintain an accurate and updated planning RUG for each resident. As shown, in step 162 of the method 160, resident data about a resident's needs, schedules, and/or RUGs level is received. This information can be received with other resident data as previously described. The method 160 can then determine, in step 164, which RUGs level or levels are applicable to the resident. These levels can each be presented to the user via a user interface as shown in FIG. 10.

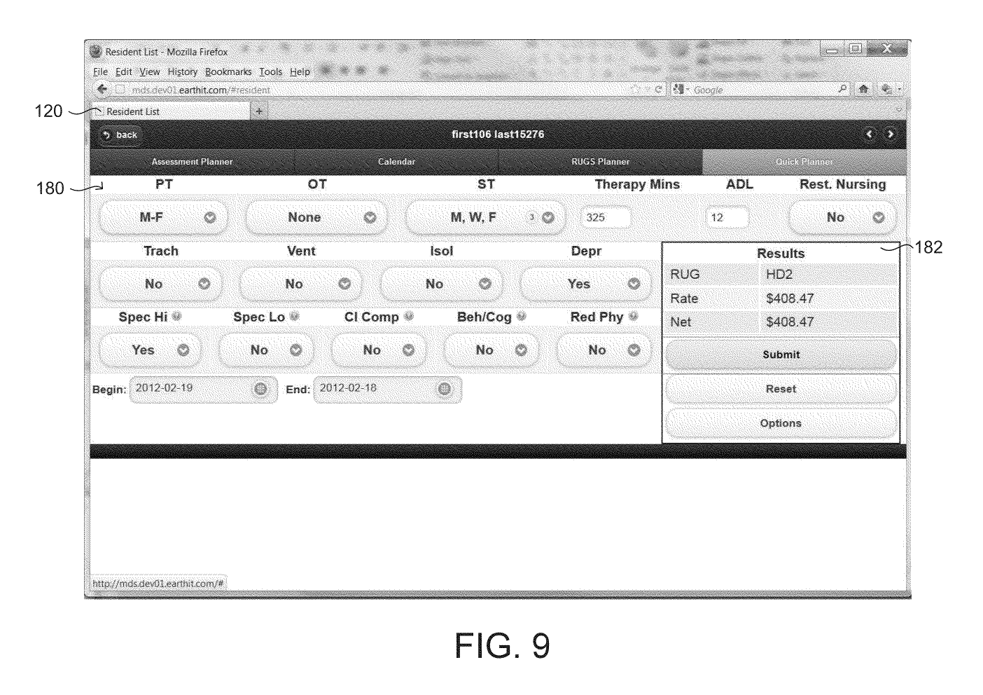

Turning now to FIG. 9, a “Quick Planner” site can be provided to the user in order to receive information about a resident treatment and to provide a planning RUG most applicable to the level of care that the Interdisciplinary Team decides is in the best interests of the resident. For example, this site can provide a user interface 180 through which a user can insert or select parameters of the residents treatment and conditions of the resident. For example, via the user interface 180, a user can select the number of days a week in which the resident should engage in physical therapy (PT), occupational therapy (OT), speech therapy (ST), along with the number of minutes in which the resident is engaged in each therapy activity. Additionally, the user can enter the resident ADL score and if restorative nursing (Rest. Nursing) is prescribed to the resident.

Continuing the example, the user can indicate if the resident needs extensive services, such as treatment for depression (Depr) or other conditions (e.g., Trach, Vent, Isol). Moreover, in some embodiments, the user can indicate the nursing conditions applicable to the resident, which are shown as Spec Hi, Spec Lo, CI Comp, Beh/Cog, and Red Phy. Lastly, the user can indicate the date range for which this treatment or these conditions are applicable. Upon receipt of this information, the system can compute results 182, which include the applicable planning RUGs level, based on the input from the Interdisciplinary Team. As shown in FIG. 10, an alternative Nursing RUGs level is shown to the Therapy RUGS.

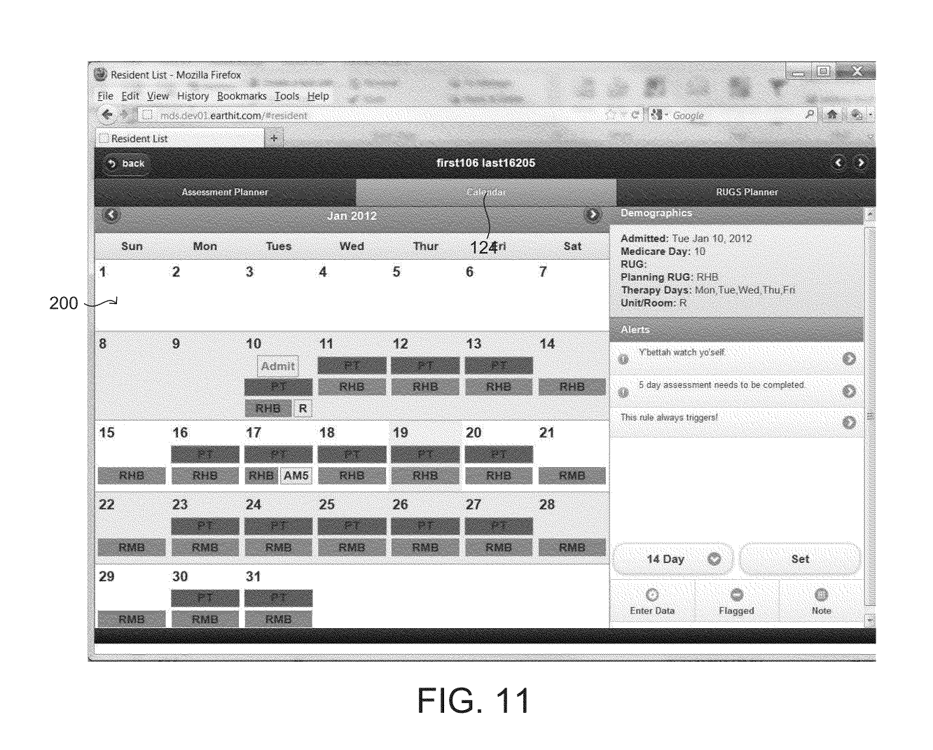

Reference will now be made to FIG. 11, which illustrates a calendar site 124 on the resident data site 120, which provides the user with a calendar graphic 200 of the residents care and therapy schedule during a time period. In some configurations, the calendar items on the calendar graphic 200 can be selected to bring up a user interface that provides details about each calendar item, and/or permits the user to insert data or reports about these activities or modify this calendar item such as the planning RUG.

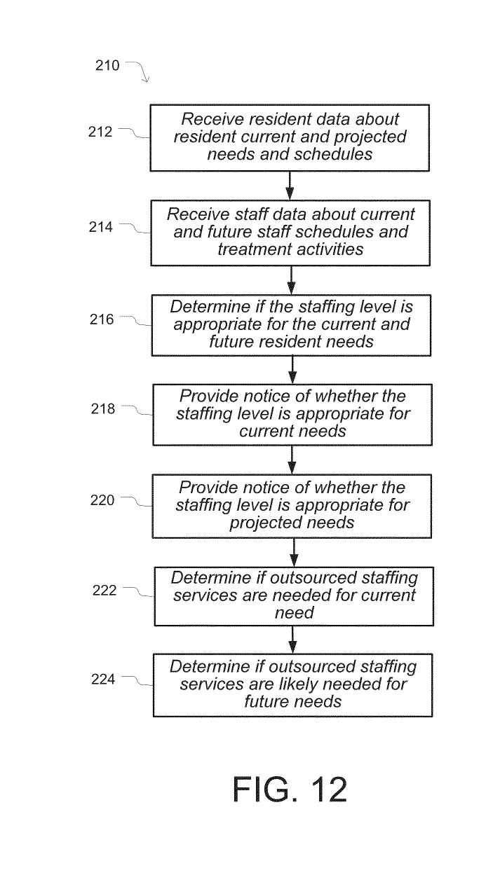

From the foregoing, it will be seen that the present systems and methods can enable an administrator of a LTC facility to view specific data about each resident; recognize if care or therapy, or reports about the residents trigger a particular rule or indicate a failure to follow the care plan; and make informed team-based decisions on the level of care best for the resident. Additionally, in some embodiments, the system can provide an administrator with additional tools for managing a LTC facility, which do not directly relate to the residents. These tools can assist the administrator in managing the facility staff and staffing levels, training of staff, facility budgeting and financial forecasting, and the preparation of meeting agendas and reports. These tools can also utilize rules data to analyze whether activities of the facility or staff trigger one or more rules. Each of these additional tools will now be described with reference to FIGS. 12 through 15.

Turning to FIG. 12, as previously mentioned with reference to FIG. 3, in some embodiments the system receives staff data relating to the therapy and care activities for the residents by the staff of the LTC facility. This information can include information about the staff schedules and availabilities. Using this information for each or some of the staff members, the system can identify if the facility is operating at an appropriate staffing level in order to determine if the facility has too many staff, not enough staff, and/or needs to use outsourced staff in order to meet its needs. Accordingly, in some embodiments, a method 210 is provided by the system for assessing staffing level of the facility. In steps 212 and 214, resident and staff data is received. The resident data can identify current and projected care and therapy needs and schedules for each resident. The staff data can identify current and future (or projected) care or treatment schedules and activities for each staff member. In step 216, the method 210 can determine if the staffing level is appropriate for the current and future resident needs. This determination can match care and therapy needs with available staff to fill the need. This determination can also identify instances where staff is not utilized or is needed. Following this determination, step 218 of the method 210 can provide the user with notice of whether the staffing level is appropriate for current needs as well as for projected needs. Correspondingly, in steps 222 and 224, the method can determine and/or notify the user if outsourced staffing services are needed for current or future needs. In some embodiments, local rules data in the rules module 60 or local rules module 50 (of FIG. 2) identifies the acceptable or ideal staff activities needed to fill one or more particular resident needs.

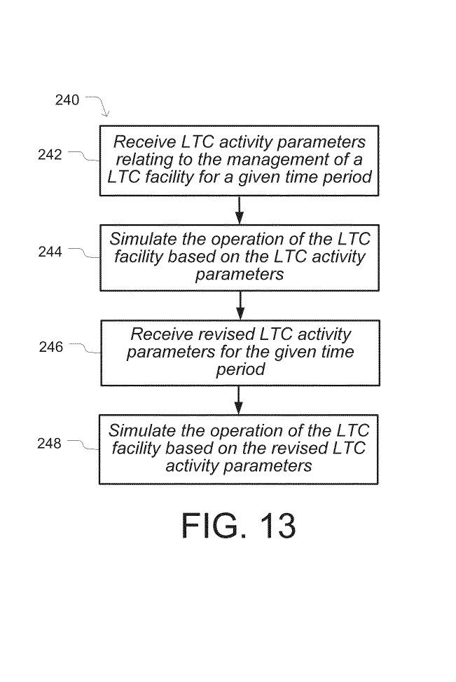

In some embodiments of the method 210, the system is configured to analyze current resident data and to provide a projection or estimate of future resident care and therapy needs. These projections can be utilized to project staffing needs, as described above, as well as to project future income from reimbursements, future expenditures, and other future sources of income. Turning now to FIG. 13, training resources provided in method 240 allow a facility administrator to simulate the operation of LTC facility under various conditions. The simulation may be provided by the training module 68 (of FIG. 2), and can be configured to simulate the operation of the LTC facility based on real or fictitious facility parameters. For example, as shown in step 242, the method 240 can receive facility activity parameters relating to the management of the facility. The simulation can be run over a specific period of time or for an indefinite future period. In step 244, the method 240 can simulate the operation of the facility based on the received parameters. After reviewing the results of the simulation, the administrator may revise the activity parameters in step 246, and re-run the simulation, in step 248. This ability can enable the facility administrator to better understand the operation of the facility, to optimize the performance of the facility, and to anticipate how to respond to various changes or circumstances that may arise.

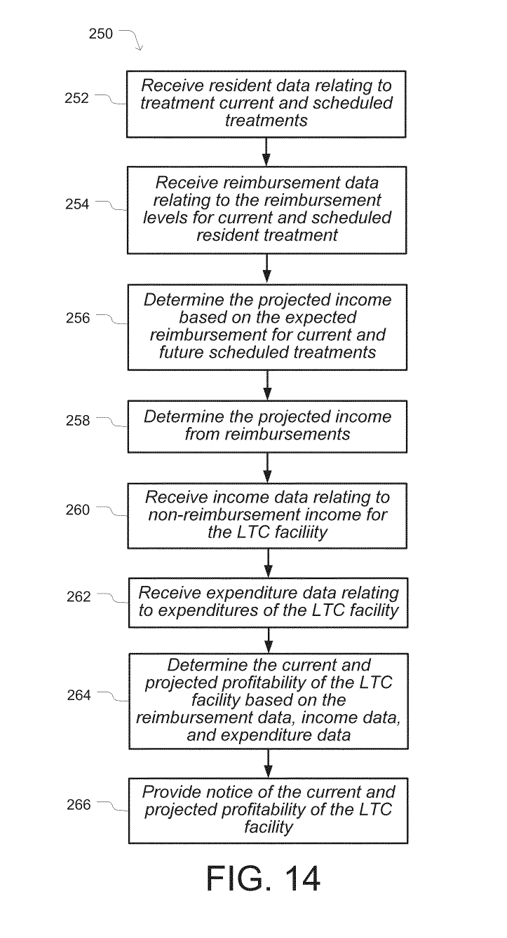

Turning now to FIG. 14, some embodiments of the system may be useful in projecting and calculating the financial condition of a LTC facility. Such systems and methods can identify the amount of money that will be reimbursed to the facility for current and projected care and therapy activities. The system can also recognize other sources of income and provide this information to facility managers to calculate profitability of the facility. An example of such a method 250 is shown in FIG. 14. In step 252, resident data is received that relates to the current care planned treatment schedules for facility residents. In step 250, reimbursement data that relates to the reimbursement levels for these treatments is received or accessed. Next, in step 256, the method 250 determines the projected income based on the expected reimbursements for these current and future scheduled treatments. In step 258, projected income from reimbursements is determined. In step 260, the method 250 can optionally receive income data relating to non-reimbursement income. This income can relate to other fees or income from other services provided by the facility. In step 262, the method 250 can receive expenditure data relating to expenditures of the facility. In step 264, the method 250 can determine the current and projected profitability of the facility based on the reimbursement data, the income data, and the expenditure data. This information can be provided to a user via a user interface or other mechanism, in step 266. For example, in some embodiment, this financial information can be provided via a dashboard user interface that graphically illustrates reimbursement income, non-reimbursement income, expenditures, and/or profitability.

Referring again to FIG. 4, in some embodiments the website or other user interface can provide a report section 106. The report section can be utilized to generate pre-meeting reports 108 or agendas and post meeting reports 110. These meetings can include program planning session (PPS) meetings. These reports can be used to increase efficiency at such meetings. In particular, the reports can include detailed information regarding various aspects of operating the facility such as the status or treatments of individual residents including compliance of previous or proposed treatments with governing rules or regulations such as Medicare or Medicaid, information regarding the staff, or any of the other types of information discussed above. This information can be provided in a quickly accessible and easily understood format for use during the meeting. This format can display the most relevant information for a particular resident in a single, or few, user interfaces where an administrator or other staff member can quickly determine which actions need to be taken with regards to a particular resident or group of residents.

In a particular example, during such meetings, the system, in addition to providing an indication regarding the actual care given to the resident, can also provide an indication of any data (e.g. the filling out of forms or progress notes) that may be required to receive reimbursement for the care provided or to be provided. The system can also provide suggested additional treatments or alternative treatment plans that may be considered during the meeting that may be more beneficial for a resident. The system can determine and display such alternatives to facilitate the discussion of which treatments or care will be provided to a resident in the future.

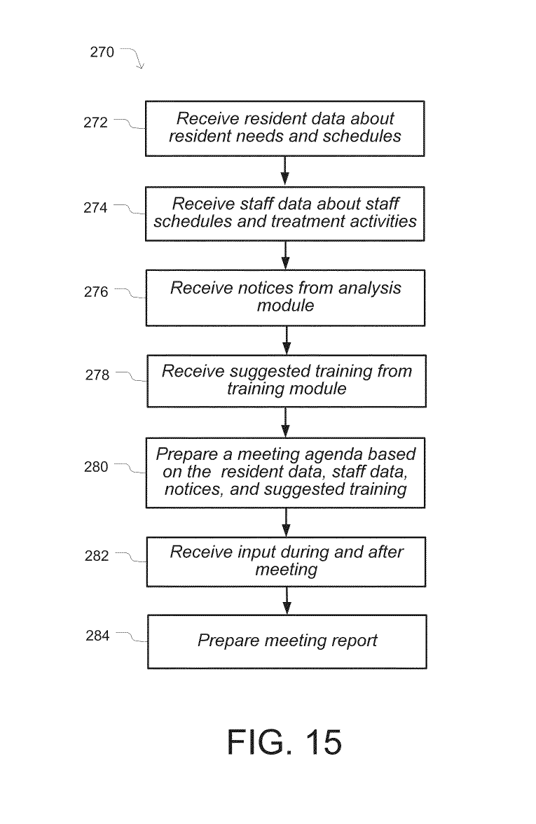

As such meetings are conducted, action items can be identified to the system which can then generate a post meeting action report that includes a listings of the identified action items along with any other information to assist in the completion of the action items (e.g. who is assigned to perform/manage the action item, a timeframe for completing the action item, etc.). A method for generating these reports is shown in FIG. 15.

In FIG. 15, a method 270 is shown for generating pre- and post-meeting reports from data available in the system. The ability to prepare these reports may facilitate and simplify and administrator's task in preparing for a staff meeting. As shown, in step 272, the method 270 can receive resident data about resident needs and schedules. In step 270, the method 270 can receive staff data about stuff schedules and treatment activities. This information can provide the administrator with an understanding of what care and therapy is needed for each resident. This information can also educate the administrator as to what staff members will be performing this care and this therapy. Moreover, in some embodiments, in step 276, the method 270 can also receive notices, alerts, or triggered rules from the analysis module 66 (in FIG. 2). The information from the analysis module can indicate if rules are being triggered or may be triggered, can suggest modified care plans for facility residents, and can identify other triggering of general rules or local rules. In addition, in step 278, the method 270 can receive suggested training from the training module 68 (in FIG. 2), such as that described with reference to FIG. 13.

After receiving at least some of this information, in step 280, the method 270 can prepare a meeting agenda for a staff meeting such as a PPS meeting. Some staff meetings can gather together the facility administrator, the director of nursing, outsourced therapists, case managers, social service providers, recreational therapists, dietary specialists, and/or other staff. During or after the meeting, the administrator or another user can optionally input modifications to the staff data or resident data for modification of the planning RUG based on the content of the meeting. Thereafter, in step 284, the method 270 can prepare a meeting report based on the modified inputs, the original inputs, and the pre-meeting agenda. In some embodiments, the method 270 further submits or stores these reports or agendas in a database.

From the foregoing, it will be seen that, the systems and computer implemented methods can provide software resources that can compile information about residents, staff, and regulations and provide this information to the administrator. Moreover, the systems and methods can analyze the compiled information to ensure that residents are receiving proper care, staff is optimally utilized, regulation and rules are satisfied, and/or the facility is operating within its available budget from reimbursements. The systems and methods may also provide or suggest alerts, tips, tools, and training to the administrator and/or facility staff. These resources can also assist administrators in managing the increased pressure on administrators to efficiently manage the staff and care and to ensure financial stability of their facility.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. At a computer system, a method for aiding in the management of a long-term care (LTC) facility, the method comprising:
receiving rules data relating to rules for operating a LTC facility;
receiving resident data relating to therapy and care needs of residents of the LTC facility;
analyzing the resident data in light of the rules data; and
displaying, using a user interface, notifications that identify one or more of the rules triggered based on the resident data.

2. The method of claim 1, wherein the rules data include one or more of Medicare regulations, Medicaid regulations, and CMS rules.

3. The method of claim 2, wherein the rules data include RUGs level standards and the resident data includes a current RUGs level, and the method further comprises identifying and displaying planning RUGs levels for a resident based on the resident data and the staff data that relates to the therapy and care activities for the resident.

4. The method of claim 1, further comprising, based on the resident data, preparing a meeting agenda for an administrator of the LTC facility for a meeting with LTC staff.

5. The method of claim 4, further comprising:
receiving updated resident data; and
preparing a post-meeting report based on the updated resident data and staff data and the meeting agenda.

6. The method of claim 1, further comprising:
receiving staff data relating to the therapy and care activities for the residents by staff of the LTC facility; and
analyzing the staff data in light of the rules data; and
displaying, using the user interface, notifications that identify one or more of the rules triggered based on the staff data.

7. The method of claim 6, wherein the staff data includes current staff therapy and care schedules, and wherein the resident data includes current resident care and therapy needs, and wherein the rules data defines an appropriate staffing level based on staff therapy and care schedules and resident care and therapy needs, and wherein analyzing the resident data and the staff data in light of the rules data includes determining if the LTC staff has an appropriate staff level for current resident care and therapy needs.

8. The method of claim 7, further comprising:
projecting future resident care and therapy needs based on the resident data; and
determining if the LTC facility is projected to have an appropriate staff level for projected future resident care and therapy needs.

9. The method of claim 8, determine if outsourced staff services are needed to fill current or future staffing needs.

10. The method of claim 6, wherein the staff data includes records relating to therapy and care activities, and wherein the rules data includes rules regarding the therapy and care activities, and the method further comprising determining a level of compliance based on whether the records relating to the therapy and care activities comply with the rules regarding the therapy and care activities.

11. The method of claim 11, further comprising providing teaching materials relating to increasing the level of compliance if the level of compliance falls below a predetermined level.

12. The method of claim 1, further comprising:
receiving reimbursement data relating to the reimbursement levels for therapy and care activities for the residents by staff of the LTC facility; and
determining a projected income from reimbursements based on reimbursements data, staff data, and resident data.

13. The method of claim 12, further comprising:
receiving expenditure data relating to expenditures of the LTC facility; and
determining current and projected profitability of the LTC facility based on at least the reimbursement data and expenditure data.

14. The method of claim 1, further comprising simulating the operation of the LTC facility for a time period based on LTC activity parameters, which include resident data, rules data, and staff data.

15. The method of claim 14, further comprising:
receiving revised LTC activity parameters; and
simulating the operation of the LTC facility based on the revised LTC activity parameters.

16. The method of claim 1, further comprising projecting occupancy of the LTC facility at a future date based on the resident data.

17. The method of claim 1, further comprising displaying, using a user interface, resident data relating to the therapy and care needs of the residents of the LTC facility.

18. A system for aiding in the management of a long-term care (LTC) facility, the system comprising:
a rules module configured to receive and store rules data relating to rules for operating a LTC facility;
a resident care module configured to receive, from a LTC computer system, and store resident data relating to the therapy and care needs of residents of the LTC facility;
an analysis module configured to analyze the rules data and the resident data to identify whether one or more of the rules are triggered based on the resident data.

19. The system of claim 18, further comprising a staff activity module configured to receive, from a LTC computer system, and store staff data relating to the therapy and care activities for the residents by staff of the LTC facility, and wherein the analysis module is configured to analyze the rules data and the staff data to identify one or more of the rules that may be triggered based on the staff data.

20. The system of claim 18, wherein the system is a server system configured to transmit a notice of the one or more rules that are triggered to a mobile computer for display via a user interface.
